(19) United States
(12) Patent Application Publication
(10) Pub. No.: US 2026/0348995 A1
(43) Pub. Date: Nov. 5, 2026

(54) DEVICES, METHODS, AND SYSTEMS FOR AUTOMATION OF MEASUREMENTS IN THE CONSTRUCTION MATERIAL TESTING INDUSTRY

(71) Applicant: InstroTek, Inc., Research Triangle Park, NC (US)

(72) Inventors: Ali Regimand, Raleigh, NC (US); Thomas Brock, Durham, NC (US); Rich Stacy, Fayetteville, NC (US); Ethan Steckmann, Youngsville, NC (US); Tallispfor Cobb, Raleigh, NC (US)

(21) Appl. No.: 19/667,817

(22) Filed: May 5, 2026

Related U.S. Application Data
(60) Provisional application No. 63/799,957, filed on May 5, 2025.

(51) Int. Cl. G01N 35/00; G01N 33/42

(52) U.S. Cl. CPC G01N 35/00871; G01N 33/42; G01N 2035/0091

(57) ABSTRACT

Some embodiments of the present invention are directed to a method for automating measurements for construction material testing, the method including: connecting an interface module to test equipment and to an electronic device; displaying, using a display associated with the electronic device, a test selection menu including a plurality of different tests for testing construction material; selecting, using an input mechanism associated with the electronic device, one of the plurality of different tests; measuring, using the test equipment, one or more physical properties of the construction material for the selected test; transmitting, using the interface module, the one or more physical properties from the test equipment to the electronic device; and automatically determining, using a data analysis application, results of the selected test based on the one or more physical properties.

### REFERENCE TO PRIORITY APPLICATION

This application claims priority from U.S. Provisional Patent Application No. 63/799,957, filed May 5, 2025, with the United States Patent and Trademark Office, the disclosure of which is incorporated by reference herein in its entirety.

### BACKGROUND

Traditionally, operators have recorded test measurements and made calculations manually off these measurements. This process introduces potential errors and requires extensive oversight by laboratory supervisors to ensure accuracy.

### SUMMARY

Some embodiments of the present invention are directed to a method for automating measurements for construction material testing, the method including: connecting an interface module to test equipment and to an electronic device; displaying, using a display associated with the electronic device, a test selection menu including a plurality of different tests for testing construction material; selecting, using an input mechanism associated with the electronic device, one of the plurality of different tests; measuring, using the test equipment, one or more physical properties of the construction material for the selected test; transmitting, using the interface module, the one or more physical properties from the test equipment to the electronic device; and automatically determining, using a data analysis application, results of the selected test based on the one or more physical properties.

In some embodiments, the plurality of different tests include a plurality of different American Association of State Highway and Transportation Officials (AASHTO) tests and/or a plurality of different American Society for Testing and Materials (ASTM) tests.

In some embodiments, the method further includes outputting a report including the results of the selected test in response to a request using the input mechanism. The method may further include manipulating, using the input mechanism, the appearance of the report.

In some embodiments, the method further includes, after selecting one of the plurality of different tests, displaying, using the display, step-by-step instructions for carrying out the selected test, the instructions including graphics and/or text.

In some embodiments, the method further includes, after selecting one of the plurality of different tests, displaying, via the display, a list of items needed to perform the selected test, the list including graphics and/or text.

In some embodiments, the data analysis application is included on the electronic device.

In some embodiments, the data analysis application is included on the cloud connected to the electronic device.

In some embodiments, the test equipment includes a scale.

In some embodiments, the interface module is configured to connect to a plurality of different types of test equipment. The method may further include: displaying, using the display, a test equipment selection menu comprising the plurality of different types of test equipment; and selecting, using the input mechanism, one of the plurality of different types of test equipment.

Some other embodiments of the present invention are directed to a system for automating measurements for construction material testing, the system including: test equipment; a local hub comprising a display and an input mechanism; an interface module connecting the test equipment and the local hub and configured to transmit data therebetween; and a data analysis module operatively associated with the local hub and configured to: direct the local hub to display a test selection menu including a plurality of different tests for testing construction material; receive a selection of one of the plurality of different tests based on input from the input mechanism; direct the interface module to initiate the selected test using the test equipment; direct the interface module to transmit one or more physical properties of the construction material for the selected test; and automatically determine results of the selected test based on the one or more physical properties.

In some embodiments, the plurality of different tests include a plurality of different American Association of State Highway and Transportation Officials (AASHTO) tests and/or a plurality of different American Society for Testing and Materials (ASTM) tests that are preloaded on the interface module.

In some embodiments, the data analysis module is configured to generate a report including the results of the selected test in response to a request using the input mechanism.

In some embodiments, the data analysis module is configured to modify the appearance of the report in response to input from the input mechanism.

In some embodiments, the data analysis module is configured to direct the local hub to display guided instructions for carrying out the selected test, the instructions including graphics and/or text.

In some embodiments, the data analysis module is configured to direct the local hub to display a list of items needed to perform the selected test, the list including graphics and/or text.

In some embodiments, the data analysis module is configured as a data analysis application operatively associated with the local hub.

In some embodiments, the test equipment includes a scale.

In some embodiments, the interface module is configured to connect to a plurality of different types of test equipment. The data analysis module may be configured to: direct the local hub to display a test equipment selection menu including the plurality of different types of test equipment; and receive a selection of one of the plurality of different types of test equipment based on input from the input mechanism.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIGS. 1 and 2 are schematic illustrations of a system for automating measurements for construction material testing according to some embodiments of the present invention.

FIG. 3 is another schematic illustration of a system for automating measurements for construction material testing according to some embodiments of the present invention.

FIG. 4 is a flowchart illustrating example methods according to some embodiments of the present invention.

FIG. 5 is a perspective view of a kit according to some embodiments of the present invention.

FIGS. 6-8 are screenshots of an electronic device according to some embodiments of the present invention.

### DETAILED DESCRIPTION

In the construction material testing industry, traditionally, operators have recorded test measurements and made calculations manually off these measurements. This process introduces potential errors and requires extensive oversight by laboratory supervisors to ensure accuracy.

Embodiments of the present invention streamline this approach by providing a seamless method and system for direct connection to test equipment, either via cable or through radio frequency (RF) technologies. By automatically collecting the required data and transmitting it to a device (either directly or through a hub), which then performs the calculations, embodiments of the present invention automate the processes and generate comprehensive reports for review.

Embodiments of the present invention can transform lab operations, helping technicians conduct routine tests efficiently. Even if a technician is not fully trained, embodiments of the present invention can provide step-by-step test procedures to quickly capture test measurement data, such as weight, force, temperature, humidity, pressure, distance, displacement, or other measurements made as part of a test standard, from test equipment such as a scale, thermometer, pressure sensor, oven, gyratory or impact compactor, load frame, compression frame, displacement sensor, etc. found in a construction materials laboratory. Embodiments of the present invention eliminate the need to write down measurements (e.g., mass measurements) – the devices, systems, and methods described herein can collect the data, determine the test results, and report the test results.

In some embodiments, the present invention can allow users to remotely tare scales, capture sample weights, and follow guided test procedures. In some embodiments, the interface module and/or the application associated therewith may be preloaded with a plurality of different American Association of State Highway and Transportation Officials (AASHTO) tests and/or a plurality of different American Society for Testing and Materials (ASTM) tests. For example, the interface module and/or the application associated therewith may include built-in tests for AASHTO T 27 (Sieve Analysis of Fine and Course Aggregates), AASHTO T 30 (Mechanical Analysis of Extracted Aggregate), AASHTO T 166 (Bulk Specific Gravity of Compacted Asphalt Mixtures Using Saturated Surface-Dry Specimens), AASHTO T 209 (Theoretical Maximum Specific Gravity and Density of Hot Mix Asphalt), AASHTO T 255 (Total Evaporable Moisture Content of Aggregate by Drying), AASHTO T 269 (Percent Air Voids in Compacted Dense and Open Asphalt Mixtures), AASHTO T 308 (Asphalt Binder Content by Ignition Oven), AASHTO T 329 (Moisture Content of Asphalt Mixtures by Oven Method), AASHTO T 331 (Bulk Specific Gravity and Density of Compacted Asphalt Mixtures Using Automatic Vacuum Sealing Method), ASTM D2041 (Theoretical Maximum Specific Gravity and Density of Uncompacted Asphalt Mixtures), ASTM D2726 (Bulk Specific Gravity and Density of Non-Absorptive Compacted Asphalt Mixtures), and/or ASTM D6752 (Bulk Specific Gravity and Density of Compacted Asphalt Mixtures Using Automatic Vacuum Sealing Method).

Typically test results are done in a spreadsheet (e.g., an Excel document) that has been made by a lab to their specific requirements. Embodiments of the present invention can create clean and modern reports with shareable files such as PDF and data files such as Excel/CSV while allowing customers to customize these reports to match the documentation required by project owners and agencies, such as state departments of transportation (DOT). This customization allows the user to have the testing process automated and have reports that are tailored to their specific labs reporting requirements. Once a report is tailored to their preferences, it can be wirelessly synced or manually exported and loaded onto all devices running the software, ensuring consistent use of the customized format across the lab.

In some embodiments, voice commands can be used to tell the software what step of the process the user is currently on. The software will, based on the voice command, populate the correct fields in the app by pulling data automatically from the test equipment.

In some embodiments, a camera either built into a tablet or laptop or an external camera can be used to detect hand gestures, allowing users to control the screen and navigate through the testing process without needing to touch the screen. This touchless control method is particularly advantageous in maintaining clean and readable screens. Users can perform specific hand movements to advance steps, input data, and interact with the software seamlessly, enhancing the overall efficiency and user experience.

Furthermore, in some embodiments, the system can be paired with virtual reality (VR) goggles, enabling the app to transition into a virtual reality environment. This VR integration guides users through the testing process with augmented reality overlays, providing step-by-step instructions and visual cues. This immersive experience can help users complete tests accurately without prior training, as the augmented reality elements ensure they follow the correct procedures. By leveraging VR and hand gestures, embodiments of the present invention not only improve the accuracy of tests but also democratize access to sophisticated testing methods, making them accessible to operators with varying levels of expertise.

The automation provided by embodiments of the present invention can greatly reduce the risk of errors inherent in manual data recording and entry, enhancing test results' reliability. Moreover, the user-friendly interface guides operators through each step of the testing process, minimizing the need for extensive training and ensuring consistent, accurate outcomes. Ultimately, embodiments of the present invention streamline construction material testing procedures, facilitating efficient design and ensuring the quality of construction and transportation infrastructure projects.

Some embodiments include the ability to track the location of the Interface Module and/or Local Hub using a positioning technology, such as GPS, GPS RTK, surveying station equipment, etc. This information can be used to track the location where the test measurement was performed and/or the location of the device itself. This is beneficial for classifying data based on location for internal quality control and external customer support.

Embodiments of the present invention include two different hardware devices. The first hardware device is a microcontroller-based module which has a wireless RF connection (e.g., Bluetooth, BLE, Wi-Fi, etc.) or a cable interface with the test equipment (this hardware is hereafter referred to as the “Interface Module” or “Interface Device”).

The Interface Module then connects wirelessly (e.g., Bluetooth, BLE, Wi-Fi, etc.) or via a cable with an electronic device such as a desktop computer, laptop, smartphone, tablet, or similar custom hardware (hereafter referred to as the “Local Hub”) which may then be connected to a cloud database wirelessly (e.g., Wi-Fi, 4G, or 5G) or via an ethernet cable.

The Interface Module may send commands to the test equipment at the request of the Local Hub, including commands to tare the test equipment, send back the current weight reading, or request data logged by the equipment during the last run of a test. The wireless advertising ID of the Interface Module can be modified by the Local Hub. The firmware running on the Interface Module can also be updated by the Local Hub over the air. The Interface Module informs the Local Hub which test equipment manufacturers it can support, and whether communication is successful with the test equipment it is connected to. The Local Hub can set the Interface Module to interface with any test equipment from a manufacturer on this list if the Interface Module is moved to a new piece of test equipment.

The data analysis app can be run on either the Local Hub or directly in the cloud. It allows the user to select an AASHTO or ASTM construction material test standard from a menu of available standards and then guides the user through conducting the selected test by text and pictures. For example, at steps where it is necessary to take a mass reading, the user may click a button, and the weight data is loaded into the app automatically via the Interface Module. At completion, the app performs the calculations to produce the desired parameter, which is displayed on the screen, reported as a PDF file or downloaded in a spreadsheet format. The app includes a feature allowing the user to change the wireless advertising ID of the Interface Module so that it can be differentiated from other Interface Modules nearby. If a new revision of the module’s firmware has been released, the app allows the user to download it to the Interface Module via the wired or wireless connection. The app also allows the user to see which test equipment manufacturer the module is set to interface with and change it if necessary.

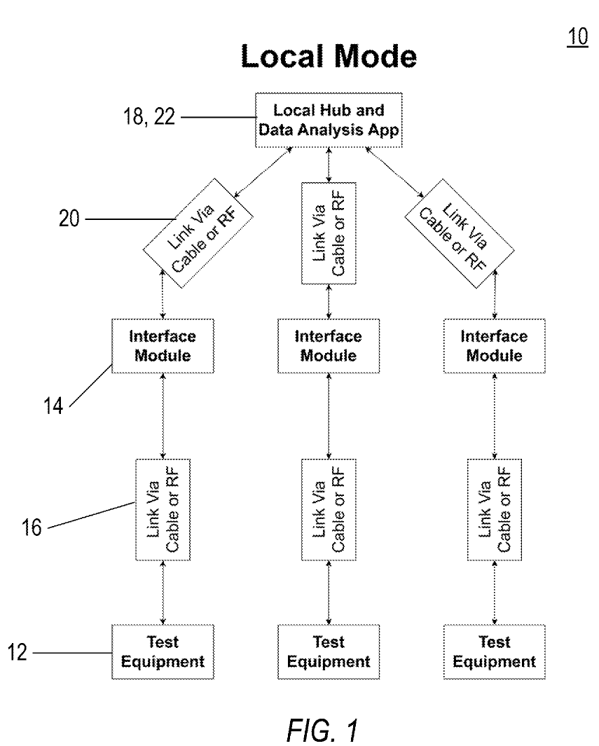

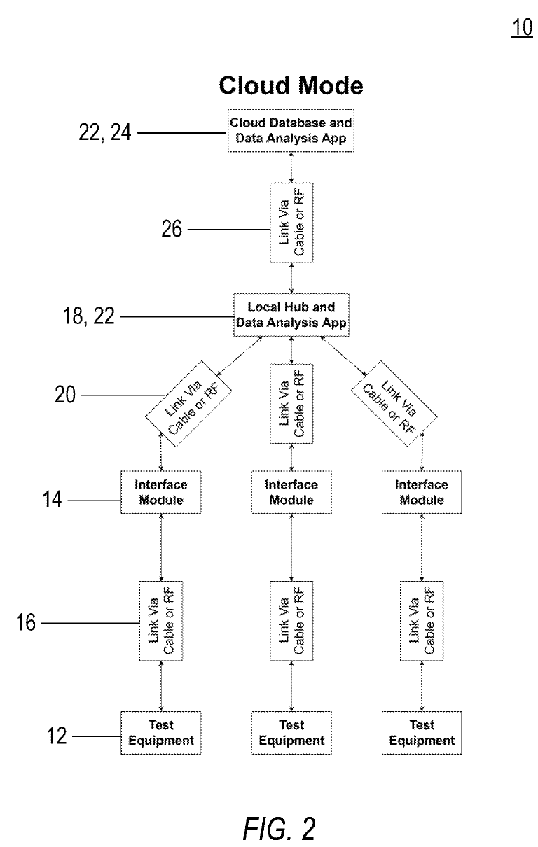

FIGS. 1 and 2 illustrate embodiments of an example system 10 for automating measurements for construction material testing. Test equipment 12 may be any test equipment capable of receiving commands and outputting data through a port such as a port on its side. In some embodiments, the test equipment 12 is or includes a scale.

The interface module 14 may connect to the test equipment 12 via a cable or RF connection 16 and to a local hub 18 via a cable or RF connection 20. The interface module 14 may send requests to the test equipment 12 at the command of the local hub 18. The interface module 14 may receive and interpret data from the test equipment 12, which may then be read by the local hub 18.

The local hub 18 may be an electronic device such as a desktop computer, laptop, tablet, smartphone, custom hardware, or any other device capable of connecting to the interface module 14. The local hub 18 may be connected to the interface module 14 by including both components in a single hardware device, or separate hardware devices for the hub and interface module. The local hub 18 can run the data analysis app 22 and/or communicate with the cloud 24 via a cable or RF connection 26 (FIG. 2). It can also handle offline data collection if internet access to the cloud is interrupted and then re-sync with the cloud when access is restored.

The data analysis program or app 22 can be software which can guide the user through the process of following a selected construction materials testing standard, automatically import data from the test equipment 12 through its connection with the interface module 14, and automatically calculate results.

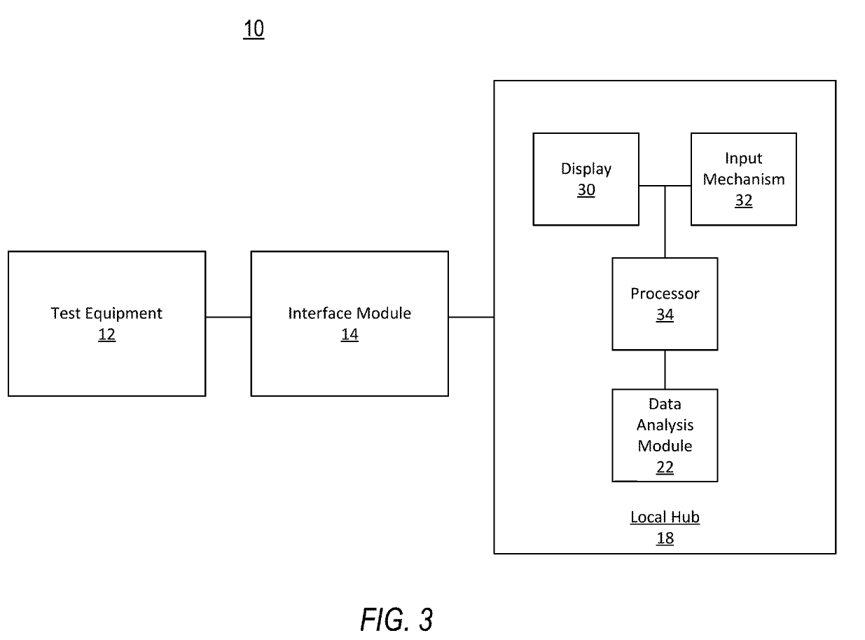

FIG. 3 is another schematic illustration of the system 10 for automating measurements for construction material testing according to some embodiments. The local hub 18 may include a display 30, an input mechanism 32, a processor 34, and a data analysis module 22, which may be configured as the data analysis application 22 described herein. As described above, the data analysis application 22 may additionally or alternatively be provided on a cloud (FIG. 2). The input mechanism 32 may be, for example, a pointing device or touch input (e.g., associated with the display 30).

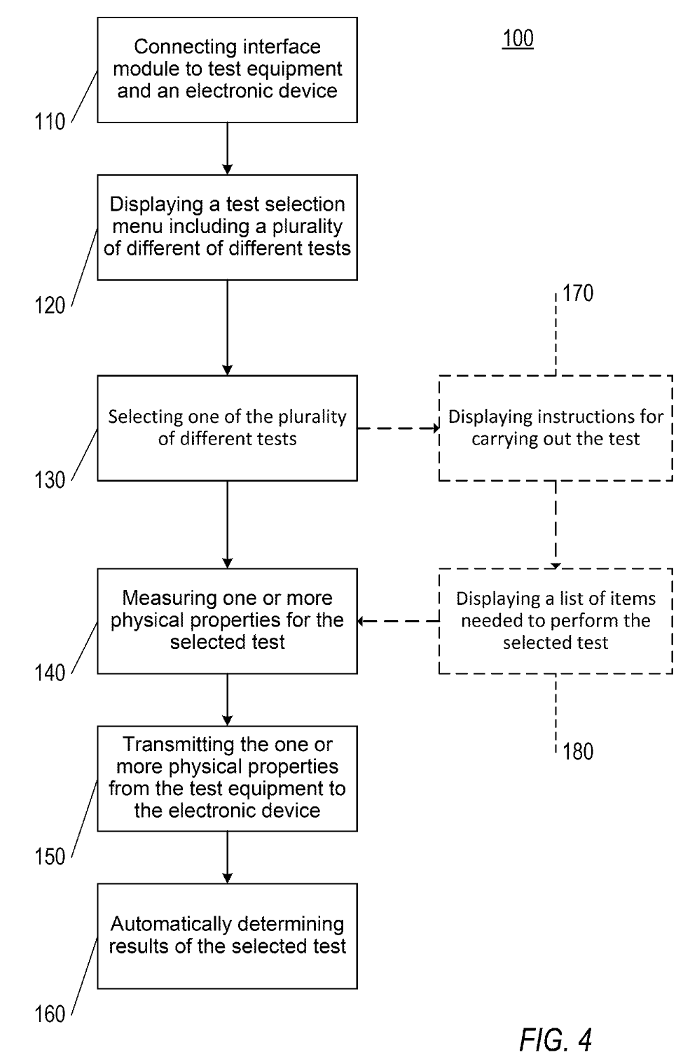

FIG. 4 is a flowchart of example methods according to some embodiments of the present invention. A method 100 for automating measurements for construction material testing may include connecting an interface module to test equipment and to an electronic device (Block 110). A test selection menu comprising a plurality of different tests for testing construction material may be displayed (e.g., using the display 30 of the local hub 18) (Block 120). One of the plurality of different tests may be selected (e.g., using the input mechanism associated with the local hub 18) (Block 130). One or more physical properties of the construction material for the selected test may be measured using the test equipment 12 (Block 140). The one or more physical properties may be transmitted from the test equipment 12 to the local hub 18 using the interface module 14 (Block 150). The data analysis application 22 may automatically determine results of the selected test based on the one or more physical properties (Block 160).

In some embodiments, guided or step-by-step instructions for carrying out the selected test may be displayed on the display 30 (Block 170). The instructions may include graphics and/or text.

In some embodiments, a list of items needed to perform the selected test may be displayed on the display 30 (Block 180). The list may include graphics and/or text.

### EXAMPLE

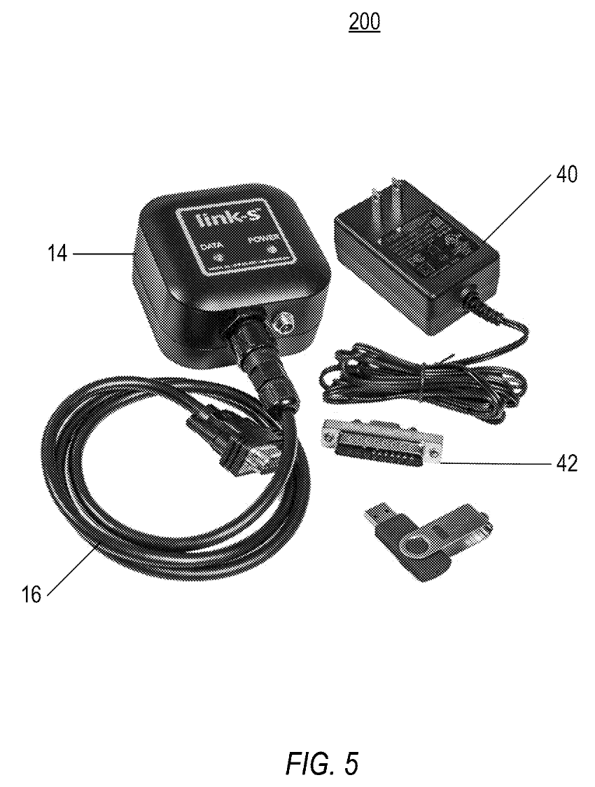

Referring to FIG. 5, an electronic mass measurement assistant (EMMA) kit or system 200 may include an interface module 14 such as a Link-S unit, an AC adapter 40, an interface cable 16, and an adapter 42 such as a 9 to 25 pin adapter.

Referring to FIGS. 1-4, the EMMA application 22 is uploaded to the local hub 18 and/or the cloud 24. The user connects the interface module 14 to the AC adapter 40 and plugs it into a 120VAC outlet. Power to the interface module could be supplied using other methods such as batteries, wireless charging, solar panels, thermoelectric generators, etc. The interface module 14 may include one or more indicators to indicate it is powered on (e.g., a red LED) and/or to indicate data is being transmitted (e.g., a green LED). The user connects the interface module 14 to the communication port of the test equipment 12 such as a scale using the interface cable 16 and adapter 42 (if needed). The user powers on the scale 12 and opens the EMMA application 22.

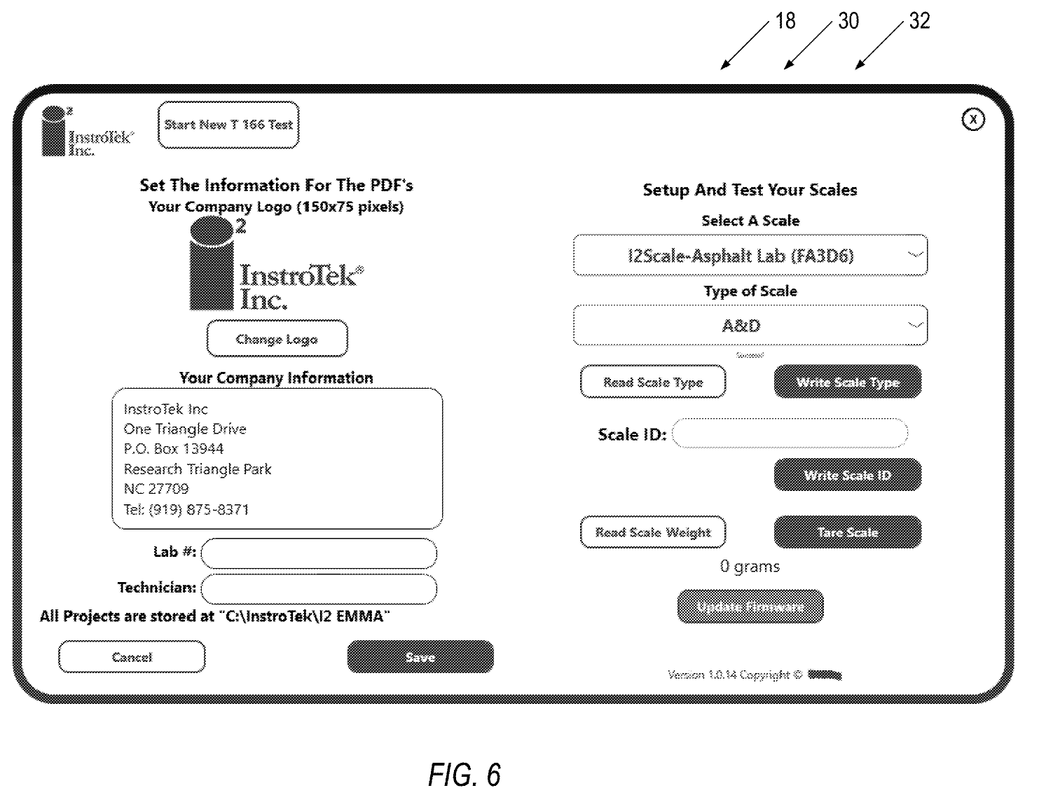

FIGS. 6-8 include screenshots of a display 30 of the local hub 18 such as an electronic device. Referring to FIG. 6, under the application settings the user can select the scale to connect to (e.g., using a drop down menu including a listing of a plurality of different scales). The user can also enter the company information, change the company logo, change the scale ID, read the current weight, and tare the scale. The user may perform these and other functions described herein using an input mechanism 32 associated with the local hub 18.

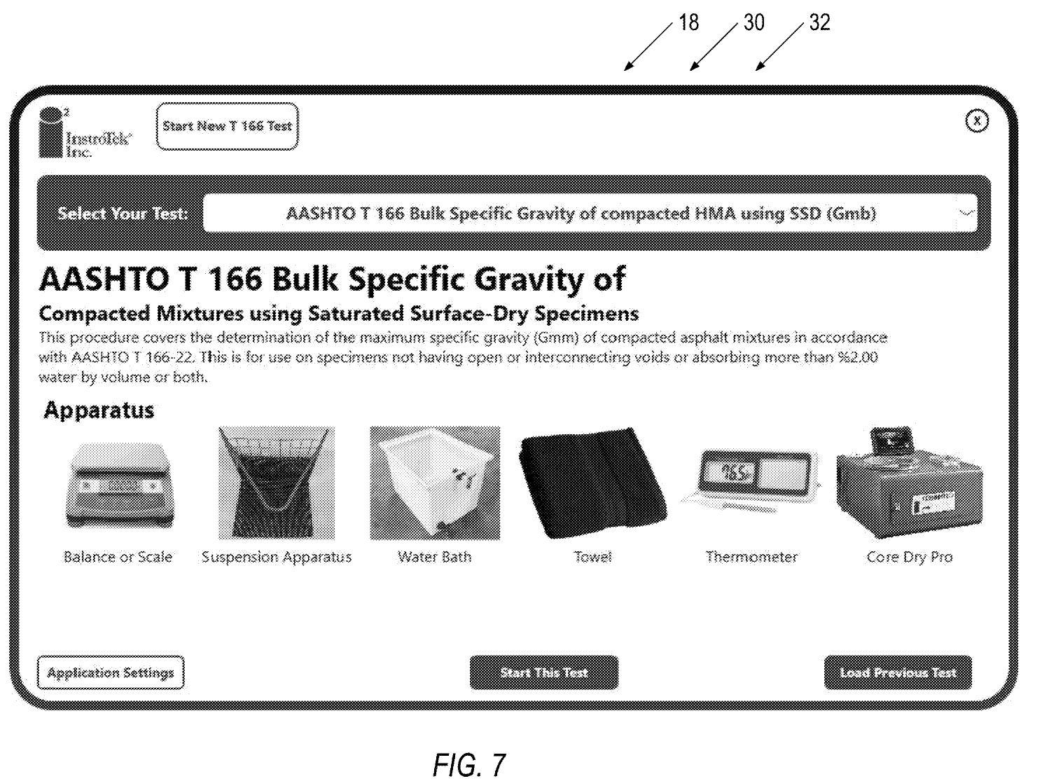

Referring to FIG. 7, the user can select the ASTM or AASHTO test to perform from the drop-down list including a listing of a plurality of different tests. After the user selects the test a list of items needed to perform the test will be automatically displayed for preparation prior to beginning the test. Once all the materials have been gathered, the user can click on the “Start This Test” button or the “Start New T 166 Test” button.

The user may then confirm and enter information for the samples. The user has the option of allowing the EMMA application to guide through the test procedure using “Guided Mode.”

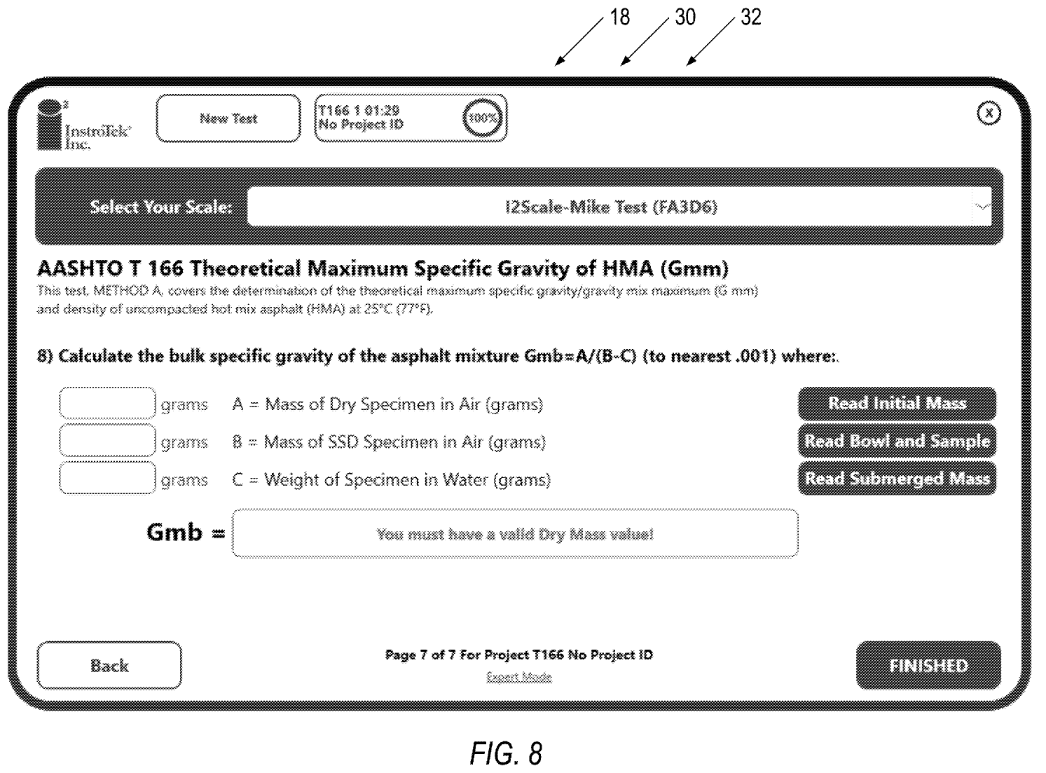

Referring to FIG. 8, the masses or weights are input. Once all the masses are entered, the test result (here, the bulk specific gravity) is calculated. The user can generate a report or repeat the test for the next sample.

Unless otherwise defined, all terms used herein have the same meaning as commonly understood by one of ordinary skill in the art. Further, all terms should be interpreted as having a meaning that is consistent with their meaning in the context of the relevant art and the present disclosure and will not be interpreted in an idealized or overly formal sense unless expressly so defined herein.

The terminology used herein is for the purpose of describing particular embodiments only and is not intended to be limiting of embodiments. The singular forms “a”, “an”, and “the” are intended to include the plural forms as well, unless the context clearly indicates otherwise. The terms “comprises”, “comprising”, “includes”, “including”, “has”, “having” and any other variations thereof specify the presence of the stated features, steps, operations, elements, components and/or groups, but do not preclude the presence or addition of one or more other features, steps, operations, elements, components and/or groups thereof.

Example embodiments may be described herein with reference to the accompanying drawings. Many different forms and embodiments are possible without deviating from the teachings of the present disclosure. Accordingly, the present disclosure should not be construed as limited to the example embodiments set forth herein. As such, it will be understood by those of ordinary skill in the art that various changes in form and details may be made therein without departing from the scope of the present disclosure. The present specification, including the drawings, shall be construed to constitute a complete written description of all combinations and subcombinations of the embodiments described herein, and of the manner and process of making and using them, and shall support claims to any such combination or subcombination.

The example embodiments are mainly described in terms of particular methods and devices provided in particular implementations. However, the methods and devices may operate effectively in other implementations. Phrases such as “example embodiment”, “one embodiment”, “embodiment” and “another embodiment” may refer to the same or different embodiments as well as to multiple embodiments. The embodiments are described with respect to systems and/or devices having certain components. However, the systems and/or devices may include fewer or additional components than those shown, and variations in the arrangement and type of the components may be made without departing from the scope of the present disclosure.

The example embodiments are also described in the context of particular methods having certain steps or operations. However, the methods and devices may operate effectively for other methods having different and/or additional steps/operations and steps/operations in different orders that are not inconsistent with the example embodiments. Thus, the present disclosure is not intended to be limited to the embodiments shown, but is to be accorded the widest scope consistent with the principles and features described herein.

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but is not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, quantum or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless (RF, optical, etc.), wireline, optical fiber cable, RF, etc., or any suitable combination of the foregoing.

Aspects of the present disclosure may be described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks. As used herein, “a processor” may refer to one or more processors.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Although some of the diagrams include arrows on communication paths to show a primary direction of communication, it is to be understood that communication may occur in the opposite direction to the depicted arrows. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Computer program code for carrying out operations for aspects of the present disclosure may be written in any combination of one or more programming languages, including an object oriented programming language such as Java, JavaScript, Scala, Smalltalk, Eiffel, JADE, Emerald, C++, C#, VB.NET, JSON, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

The above-disclosed subject matter is to be considered illustrative, and not restrictive. It will be appreciated that further variations and modifications may be made within the scope of the present disclosure. Although specific terms are employed, they are used in a generic and descriptive sense only and not for purposes of limitation.

The foregoing is illustrative of the present invention and is not to be construed as limiting thereof. The invention is defined by the following claims, with equivalents of the claims to be included therein.

We claim:

1. A method for automating measurements for construction material testing, the method comprising:
connecting an interface module to test equipment and to an electronic device;
displaying, using a display associated with the electronic device, a test selection menu comprising a plurality of different tests for testing construction material;
selecting, using an input mechanism associated with the electronic device, one of the plurality of different tests;
measuring, using the test equipment, one or more physical properties of the construction material for the selected test;
transmitting, using the interface module, the one or more physical properties from the test equipment to the electronic device; and
automatically determining, using a data analysis application, results of the selected test based on the one or more physical properties.

2. The method of claim 1 wherein the plurality of different tests comprise a plurality of different American Association of State Highway and Transportation Officials (AASHTO) tests and/or a plurality of different American Society for Testing and Materials (ASTM) tests.

3. The method of claim 1 further comprising outputting a report including the results of the selected test in response to a request using the input mechanism.

4. The method of claim 3 further comprising manipulating, using the input mechanism, the appearance of the report.

5. The method of claim 1 further comprising, after selecting one of the plurality of different tests, displaying, using the display, step-by-step instructions for carrying out the selected test, the instructions including graphics and/or text.

6. The method of claim 1 further comprising, after selecting one of the plurality of different tests, displaying, via the display, a list of items needed to perform the selected test, the list including graphics and/or text.

7. The method of claim 1 wherein the data analysis application is included on the electronic device.

8. The method of claim 1 wherein the data analysis application is included on the cloud connected to the electronic device.

9. The method of claim 1 wherein the test equipment includes a scale.

10. The method of claim 1 wherein the interface module is configured to connect to a plurality of different types of test equipment, the method further comprising:
displaying, using the display, a test equipment selection menu comprising the plurality of different types of test equipment; and
selecting, using the input mechanism, one of the plurality of different types of test equipment.

11. A system for automating measurements for construction material testing, the system comprising:
test equipment;
a local hub comprising a display and an input mechanism;
an interface module connecting the test equipment and the local hub and configured to transmit data therebetween; and
a data analysis module operatively associated with the local hub and configured to:
direct the local hub to display a test selection menu comprising a plurality of different tests for testing construction material;
receive a selection of one of the plurality of different tests based on input from the input mechanism;
direct the interface module to initiate the selected test using the test equipment;
direct the interface module to transmit one or more physical properties of the construction material for the selected test; and
automatically determine results of the selected test based on the one or more physical properties.

12. The system of claim 11 wherein the plurality of different tests comprise a plurality of different American Association of State Highway and Transportation Officials (AASHTO) tests and/or a plurality of different American Society for Testing and Materials (ASTM) tests that are preloaded on the interface module.

13. The system of claim 11 wherein the data analysis module is configured to generate a report including the results of the selected test in response to a request using the input mechanism.

14. The system of claim 13 wherein the data analysis module is configured to modify the appearance of the report in response to input from the input mechanism.

15. The system of claim 11 wherein the data analysis module is configured to direct the local hub to display guided instructions for carrying out the selected test, the instructions including graphics and/or text.

16. The system of claim 11 wherein the data analysis module is configured to direct the local hub to display a list of items needed to perform the selected test, the list including graphics and/or text.

17. The system of claim 11 wherein the data analysis module is configured as a data analysis application operatively associated with the local hub.

18. The system of claim 11 wherein the test equipment includes a scale.

19. The system of claim 11 wherein the interface module is configured to connect to a plurality of different types of test equipment, and wherein the data analysis module is configured to:
direct the local hub to display a test equipment selection menu comprising the plurality of different types of test equipment; and
receive a selection of one of the plurality of different types of test equipment based on input from the input mechanism.
